(19) United States
(12) Patent Application Publication
(10) Pub. No.: US 2005/0020978 A1
(43) Pub. Date: Jan. 27, 2005

(54) Safety device for an infusion pump

(76) Inventor: Marc Vollenweider, Adlikon (CH)

Correspondence Address: BROWDY AND NEIMARK, P.L.L.C.;624 NINTH STREET, NW, SUITE 300, WASHINGTON, DC, 20001-5303, US

(21) Appl. No.: 10/494,761

(22) PCT Filed: Nov. 5, 2002
(86) PCT No.: PCT/CH02/00594

(30) Foreign Application Priority Data
Nov. 15, 2001 (CH) 2097/01
Mar. 15, 2002 (CH) 449/02

(51) Int. Cl. A61M 1/00

(52) U.S. Cl. 604/151

(57) ABSTRACT

A throughflow locking element (20, 30) for an infusion pump receives a flexible infusion tube and is provided with a clamp valve (20; 21, 22). The clamp valve (20) has two openings (23, 24) through which the flexible infusion tube can be introduced. Said clamp valve has a lockable closed position and an open position in relation to the throughflow through the flexible infusion tube and can be inserted in a holder (30) in the infusion pump in a positive fit. A drive element (31) is provided in the infusion pump, enabling the clamp valve (20) to be moved from the open position into the lockable closed position and vice-versa, whereby the throughflow locking element (20) is sealed in a secure manner prior to removal of the flexible infusion tube from the infusion pump.

The invention relates to a safety device for an infusion pump with which liquids can be delivered via an infusion tube.

Infusions are routine procedures in medical treatments. They run for quite long periods of time. To perform such an infusion, there is, on the one hand, the purely mechanical solution of conveying the infusion liquid into the body of the patient by force of gravity. For this, mechanical dosing elements are available, for example valves fitted directly on the tube and clamping said tube. Since these act directly on the tube, the physician or nurse carrying out the treatment is able to control the flow rate by adjusting this element.

Alternatively, there is also the possibility of controlling an infusion with the aid of what are referred to as infusion pumps, which also afford the possibility of controlling the flow rate as a function of time and can also emit alarm signals via sensors.

In this case, the tube, with the infusion bag at one end and, if appropriate, with said dosing element fitted on the tube, is introduced into the infusion pump, whereupon a mechanism suitably provided in the pump manipulates the tube in order to set and to maintain a predetermined rate of flow. Since the control of the throughflow is now effected by this appliance, the dosing element which may also be present, and which forms a manual throughflow barrier, is set to the open position, so that the control of the throughflow is effected exclusively by the infusion pump. Upon opening of the appliance and removal of the tube, however, care must be taken to ensure that said dosing element, the manual throughflow barrier, is activated, since otherwise the infusion liquid runs directly into the patient without control and at what may be a high pressure.

To solve this problem of infusion pumps, a disk-shaped plastic part is known which has a longitudinal slit. Provided at one end of the longitudinal slit there is an opening, for example a circular opening, which has a greater diameter than the narrow width of the longitudinal slit. The tube is first fitted, or has been fitted in advance, through the round opening. Upon insertion of the tube into the infusion pump, this disk-shaped plastic part is fitted into and forcibly locked in a corresponding complementary recess in the infusion pump. Upon removal of the tube, this is also moved forcibly with a first applied force into the narrower longitudinal hole. Only when this has been done can the disk-shaped plastic part be removed from the holder, with an applied force greater than the force first applied. Thus, by squeezing of the tube, a throughflow of the remaining infusion liquid under the force of gravity is safely suppressed.

These devices from the prior art are complicated to use and are not 100% reliable.

In addition, these elements do not prevent incorrect insertion of the tube, in the sense of the pump conveying liquid counter to the infusion direction.

Finally, even in elaborate devices according to the prior art, free flow of a medicament into the patient can arise if the door itself is not leaktight, for example because the force is not sufficient, or is no longer sufficient, to squeeze the equipment shut. The mechanism may age or become soiled, with the result that the infusion equipment is not correctly squeezed shut. In addition to the aforementioned incorrect insertion of the infusion equipment, it is also possible for the wrong infusion equipment to be inserted, for example one that is too hard or too thin, so that the closing door cannot correctly close the tube.

Because of the existing tolerances, such problems may also not always be reliably detected by drop detectors; the same of course also applies to throughflow sensors, which are usually less precise anyway.

In order to go at least some way to tackling these problems, the pressure ratios of the pump doors of such infusion appliances are typically checked on a regular basis, for example every 24 months. Thus, as time passes from the last inspection date, uncertainty increases concerning the functional reliability of the equipment.

Starting out from this prior art, it is an object of the invention to make available an improved safety device for an infusion pump.

According to the invention, this object is achieved by the features of claim 1.

With such a design of the infusion pump, it is possible to build up a pressure in a controlled manner in the closed-off segment between the pump mechanism and the element closing the infusion tube, to monitor the extent and development of the pressure in the control device, and, in the event of operating errors, which may arise from a deviation of calibration curves for example, to suppress the functioning of the infusion pump and generate a corresponding output message.

In an advantageous embodiment of the device, the closing element, a squeezing element, can be inserted into a holder in the infusion pump and is further characterized in that the squeezing element provided on the infusion tube is a clamp valve which has a lockable closed position, in that the clamp valve can be inserted with a positive fit into the holder in the infusion pump, and in that a drive element is provided in the infusion pump, with which drive element the clamp valve can be moved from the open position into the lockable closed position and vice versa.

Through the forced release of the lockable position upon opening of the infusion pump, either electrically or mechanically, it is possible to ensure that, irrespective of the position of the manual dosing element usually also present, the inflow of infusion liquid is reliably suppressed with the opening of the infusion pump and thus already before removal of the tube.

The additional check of the squeezing element for tightness, as provided for by the safety device, advantageously takes place after each closure of the door of the infusion pump.

In an advantageous embodiment, the valve is configured asymmetrically, which ensures that it is always inserted into the infusion pump in the correct direction, so that the direction of installation of the tube is always correct.

The opening and closing are advantageously effected by an eccentric cam, which is driven mechanically, so that the function of the throughflow barrier can be checked easily. In the case of a passive infusion pump, it is also possible for the closure action to be triggered by the opening of the appliance.

The invention is explained in more detail below on the basis of a preferred illustrative embodiment and with reference to the attached drawings, in which:

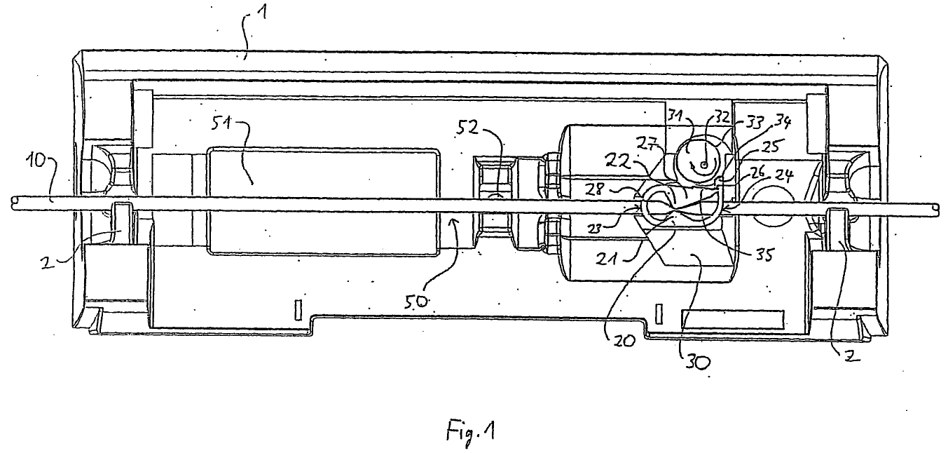

FIG. 1 shows a diagrammatic front view of an open infusion pump with inserted tube,

FIG. 2 shows a perspective view of a portion of this infusion pump with a throughflow barrier in the open state,

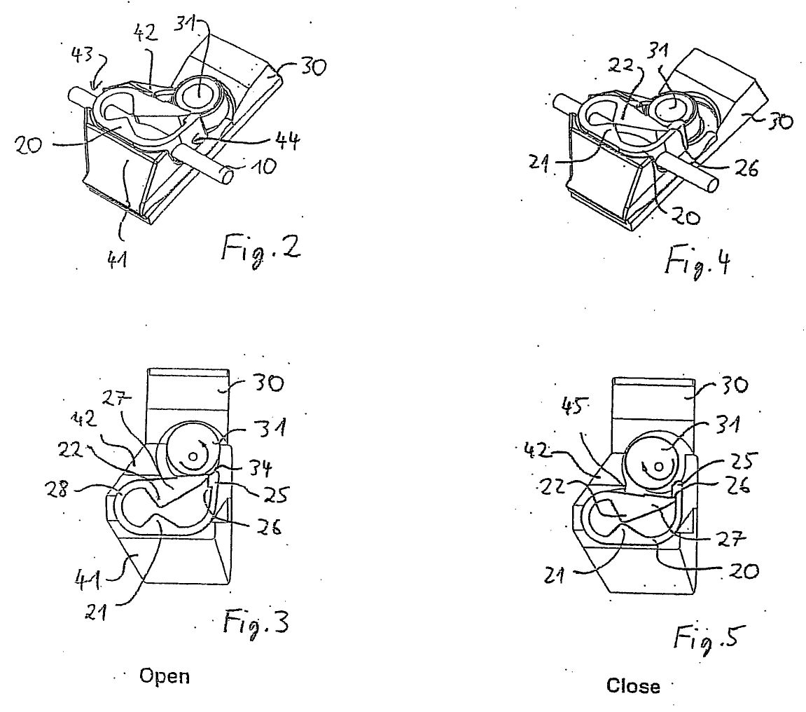

FIG. 3 shows a plan view of the throughflow barrier from FIG. 2,

FIG. 4 shows a perspective view of the throughflow barrier according to FIG. 2 in the closed state, and

FIG. 5 shows a plan view of the throughflow barrier from FIG. 4.

FIG. 1 shows a diagrammatic view of an opened infusion pump 1, that is to say of an infusion pump 1 with opened lid. This pump usually has, on both sides, a retaining hook 2 in which, with the lid opened as in FIG. 1, an infusion tube 10 can be inserted. A throughflow barrier 20, representing a squeezing element, is fitted on-the infusion tube 10.

The throughflow barrier 20 is shown in the closed state in FIG. 1, as can be seen from the fact that two opposite squeezing flanges 21 and 22 press the tube 10 together so that it is closed. As will be explained in more detail with reference to the subsequent figures, the throughflow barrier 20 has a bore 23 at one end and a bore 24 at the other end, through which bores the non-deformed tube 10 is guided. One end 25 of the throughflow barrier has a locking hook 26 behind which the other end 27 of the throughflow barrier 20 can be locked. This end 27 is flexible by means of the curved portion 28 with the aperture 23 and can be pressed until the tube 10 is squeezed together via the flange 22 on said end 27.

The throughflow barrier lies with a positive fit in a throughflow barrier holder 30 provided in the infusion pump 1. Since the throughflow barrier holder 30 is shaped corresponding to the outer shape of the uncompressed (=open) throughflow barrier 20, the throughflow barrier 20 can be inserted only in the direction shown in FIG. 1, which ensures that the direction of installation is correct. In the direction turned through 180 degrees, the free end 25 would not be able to be inserted in the part provided for the rounded portion 28.

In addition to the apertures for insertion of the tube 10, the throughflow barrier holder 30 has a further lateral opening in which a cylindrical element 31 rotates in an eccentric position about an axis 32. The cylinder jacket 33 of the eccentric disk 31 presses at one side on an end face 34 of the end 25 of the throughflow barrier 20 in order to release the engagement of the locking hook 26 and open the valve. Upon further rotation of the disk 31 in the clockwise direction, the jacket 33 then presses more and more on the rear face 35 of the other free end and presses this down so that the flanges 21 and 22 squeeze the tube 10, and the locking hook 26 finally locks into the end 25.

The functional elements of the infusion pump 1 which control the throughflow are not themselves the subject of the invention. An important feature, however, is the function of the infusion pump 1 upon opening of the lid of the infusion pump 1. The lid is connected to the control of the eccentric disk 33 to the extent that, upon opening of the lid of the infusion pump 1, this disk 31 automatically moves to the closed locking position shown in FIG. 1.

In another illustrative embodiment, it is also possible that, instead of the disk 31, a lever is provided which presses down the free end 27 of the throughflow barrier 20, and, in other embodiments, instead of an electric drive means for locking the valve upon opening of the lid, a spring-actuated device can also be provided which is released when the lid of the infusion pump 1 is opened. It can be pretensioned by the closure of the lid.

Moreover, a contact switch can be provided with which, upon opening of the lid of the infusion pump 1, this information is transmitted to a safety device, which is not shown in the appliance in FIG. 1, because it is provided together with the other electronics in the inside of the appliance. Reference number 51 designates the pump mechanism surrounding the tube 10. An area 50 is thus defined between this pump mechanism 51 and the throughflow barrier 20. In this area, a pressure sensor 52 is provided on the tube 10, with which the fluid pressure in said area 50 can be determined.

The safety device functions in the following way. As will be explained in the further course of the description, the throughflow barrier 20 can be set to “closed”. The pump mechanism 51 is then started by the safety device, which immediately leads to a pressure increase in the area 50, without liquid or pressure reaching the patient. The pressure increase is registered by the pressure sensor 52, which transmits the corresponding signals to the control device.

Thus, it is directly possible, right from the start, to automatically establish whether both the pump mechanism 51 and the throughflow barrier 20 are tight. If this is not the case, as may be possible on account of an insidious leaking of the equipment, the pump mechanism 51 is immediately stopped and/or is set to reverse operation, in order to prevent a pressure increase or flow of liquid in the direction of the patient.

When a certain pressure is reached or restored in the area 50, the throughflow barrier 20 can be opened in order to check its function, by means of the pressure difference now present in the area 50 being registered by the same pressure sensor 52 and the control device further operated. It is thus possible to determine whether the throughflow barrier 20 was correctly inserted and is correctly functioning. It would then also be possible to determine whether an outer clamp has been closed, which it may not have been possible to determine with certainty using the pressure increase test, or that, instead of the throughflow barrier 20 of the type described here with reference to FIGS. 2 to 5, only a normal roller clamp has been closed.

The action of the throughflow barrier 20 is explained in more detail with reference to the other views in the subsequent figures.

FIG. 2 shows a diagrammatic and perspective view of the throughflow barrier holder 30, with an inserted tube 10 on which a throughflow barrier 20 is arranged. Identical features have been given the same reference numbers in all the figures. FIGS. 2 and 3 show the open state of the throughflow barrier 20, the control of the infusion pump 1 regulating the throughflow.

FIG. 2 shows in particular the two opposite side walls 41 and 42 of the throughflow barrier holder 30 which enclose a large part of the throughflow barrier 20 with a positive fit. Reference numbers 43 and 44 designate the lateral openings through which the tube is guided through the throughflow barrier holder 30.

Finally, FIG. 2 clearly shows that, to safeguard the function of the throughflow barrier, it suffices to provide a throughflow barrier holder 30 in a standard infusion pump, as long as the eccentric disk 31 or a comparable element can be activated to check the function by opening the lid and/or by pressing a button.

FIG. 3 shows a diagrammatic plan view of the subject illustrated in FIG. 2, but without tube 10. By turning the disk 31 counterclockwise, the surface 34 of the free end 25 has been bent upward at the side, in order to open the valve. The free end 27 does not engage in the locking hook 26.

FIG. 4 shows a diagrammatic and perspective view of the throughflow barrier holder 30, with an inserted tube 10 on which the throughflow barrier 20 is arranged. FIGS. 4 and 5 show the closed state of the throughflow barrier 20, in which the tube can be removed from the infusion pump 1.

FIG. 4 shows in particular how the two flanges 21 and 22 squeeze the tube. The flanges are triangular in the plan view, with a straight edge in order to define in each case a line of squeezing tangential with respect to the tube 10.

Finally, FIG. 5 shows that, on securing_the function of the throughflow barrier, a gap 45 develops between the surface 42 and the free end 27 when, by means of the eccentric disk 31 or a comparable element, the free end 27 locks in the locking hook 26 of the other end 25 of the throughflow barrier 20.

Because of the simplicity of the one-piece throughflow barrier 20, the latter can be provided as standard on tubes 10, regardless of whether an infusion is to be performed with a manually adjustable dosing element or with an infusion pump 1. The throughflow barrier 20 does not interfere with the function of a dosing element outside an infusion pump 1, because it only recognizes the open and closed positions and, for this reason, an erroneous closure of the tube is immediately detected.

With a device and a method having the features of the attached claims, the safety of the clamp can be automatically checked by virtue of the fact that, upon each closure of the door of the device, the control device runs the abovementioned method. The clamp does not necessarily have to be a clamp mounted on the infusion equipment, but instead it can also be a shut-off device of the pump, with which normal equipment can be opened and closed.

Since the clamp valve 20 is of asymmetrical design, so that it can be inserted into the complementary holder 30 only in a specified manner in relation to the orientation of the infusion tube 10 in a longitudinal direction, it is possible to ensure the correct direction of installation of the infusion tube 10 in the infusion pump 1.

With the device according to the invention, occlusions downstream and upstream of the tube segment 50 can also be automatically established and can be indicated on displays on the infusion pump.

An important feature is the possibility, afforded by the invention, that, by inserting the infusion tube into the infusion pump, an automaticity is triggered and allowed to run with which it is possible to check important basic safety functions of the tube (in particular inside the infusion pump, but also outside the infusion pump) and pump (tube and clamp type compatible with this type of infusion pump, correctly inserted and correctly oriented).

1. A safety device (20, 30) for an infusion pump (1) with which liquids can be delivered via an infusion tube (10), characterized in that the infusion pump (1) can move the infusion tube (10) into a closed position, in that the pump mechanism, in the closed position of the infusion tube (10), can change the pressure conditions in the closed-off segment (50), and, with the aid of a sensor (52), the pressure variations in said closed-off segment (50) can be measured directly or indirectly, so that functions of the infusion pump (1) and of the infusion tube (10) can be checked with the measured values.

2. The device as claimed in claim 1, characterized in that the infusion pump (1) can move the infusion tube (10) into an open position, so that the changing pressure conditions in the then no longer closed-off segment (50) can be measured directly or indirectly with the aid of the sensor (52) or a further sensor, so that functions of the infusion pump (1) and of the infusion tube (10) can be checked with the measured values.

3. The device as claimed in claim 1 or 2, characterized in that the tightness of the infusion tube (10) in the pump mechanism can be checked as the function of the infusion pump (1).

4. The device as claimed in claim 1 or 2, characterized in that a squeezing element (20; 21, 22) is fitted on the infusion tube (10) and can be inserted in a holder (30) of the infusion pump (1), so that the infusion pump (1) can check the correct insertion of the infusion tube (10) and of the squeezing element (20; 21, 22).

5. The device as claimed in claim 4, characterized in that the squeezing element (20; 21, 22) is a directional element, and in that the holder (30) is a directional holder, so that the infusion pump (1) can check the correct orientation of the infusion tube (10) and of the squeezing element (20; 21, 22).

6. The device as claimed in claim 4 or 5, characterized in that the squeezing element (20; (21, 22) has a defined geometry, and in that the holder (30) has a defined geometry, so that the infusion pump (1) can check the correct tube type of an inserted infusion tube (10).

7. The device as claimed in one of preceding claims 4 through 6, characterized in that the element (20; 21, 22) squeezing the infusion tube (10) has at least one opening (23, 24) through which the infusion tube (10) can be guided, and which has the closed position in relation to the throughflow through the infusion tube (10) and the open position in relation to the through flow through the infusion tube (10), in that the safety device comprises a control device with which the element (20; 21, 22) squeezing the infusion tube (10) can be moved into the closed position, in that the safety device is connected to the pump of the infusion pump in order to allow controlled operation of the pump mechanism (51) surrounding the infusion tube (10), and in that, in the area (50) of the infusion tube insertable into the infusion pump (1), a pressure sensor (52) is provided between the pump mechanism (51) and the squeezing element (20; 21, 22), the output signal of which pressure sensor (52) can be transmitted to the control device.

8. The device as claimed in claim 7, characterized in that the squeezing element is designed as a clamp valve (20) which has a lockable closed position, in that the clamp valve (20) can be inserted with a positive fit (30) into the holder in the infusion pump (1), and in that a drive element (31) is provided in the infusion pump (1), with which drive element (31) the clamp valve (20) can be moved from the open position into the lockable closed position and vice versa.

9. A method for operating the safety device as claimed in one of claims 1 through 8, characterized in that the control device obtains a start signal generated by the closing of the door of the infusion pump or by a manual external starter switch, in that the control device acts on the pump mechanism of the infusion pump with a control signal so that the pressure in the area between pump mechanism and squeezing element rises in a predetermined time period and is then maintained or lowered, while at the same time data representative of the pressure prevailing in the infusion pump are transmitted from the pressure sensor to the control device with which, by means of data comparison, a result signal can be generated which is representative of the functional ability or functional inability of the infusion pump.

10. The method as claimed in claim 9, characterized in that the control device generates an open signal and transmits this to the squeezing element, in response to which signal the squeezing element is opened, and in that the pressure drop in the area between pump mechanism and squeezing element is detected by the pressure sensor after opening of the squeezing element and is transmitted to the control device in which, by means of data comparison, a result signal can be generated which is representative of the functional ability or functional inability of the opening of the squeezing element.
